(19) United States
(12) Patent Application Publication
(10) Pub. No.: US 2024/0197238 A1
(43) Pub. Date: Jun. 20, 2024

(54) POINT-OF-CARE PREDICTION OF MUSCLE RESPONSIVENESS TO THERAPY DURING NEUROREHABILITATION

(71) Applicant: UNIVERSITY HEALTH NETWORK, Toronto (CA)

(72) Inventors: Jose ZARIFFA, Toronto (CA); Sukhvinder KALSI-RYAN, Toronto (CA)

(21) Appl. No.: 18/555,339

(22) PCT Filed: Apr. 13, 2022
(86) PCT No.: PCT/CA2022/050574

§ 371 Date: Oct. 13, 2023

Related U.S. Application Data
(60) Provisional application No. 63/174,328, filed on Apr. 13, 2021.

(51) Int. Cl. A61B 5/397; A61B 5/00; A61N 1/36; G06F 18/2415

(52) U.S. Cl. CPC A61B 5/397; A61B 5/725; A61B 5/7267; G06F 18/2415; A61N 1/36003

(57) ABSTRACT

Devices, methods of using devices, and methods of training devices are provided. For example, a portable, hand-held device comprises: a sensor configured to record surface electromyography (sEMG) data for at least one muscle; a memory; and a processor configured to apply predetermined relationships between the sEMG data and reference data stored in the memory, and based on the relationships, generate a predicted recovery profile for the muscle. The device may implement algorithms trained in a functional electrical stimulation therapy (FES-T) program and/or may be used for predicting muscle recovery in the FES-T program.

### TECHNICAL FIELD

The present disclosure relates to systems, methods, and devices for predicting the responsiveness of a muscle or muscle group to functional electrical stimulation therapy (FES-T). More particularly, the present disclosure relates to systems, methods, and devices for determining correlation between electrophysiological biomarkers detected during surface electromyography (sEMG) and responsiveness to FES-T and application of the correlation as a predictive tool.

### BACKGROUND

Injuries to the nervous system may result in decreased muscle response, including reductions in reaching, grasping, or other motor functions. In some cases, such injuries can result in severe disability and limit the affected individual's ability to complete activities of daily living, integrate into the community, and enjoy a high quality of life. The degree of impairment of the limbs, for example, may be a major determinant of independence and quality of life after a neurological injury. Individuals suffering from such injuries often report that the recovery of limb function is their top priority. Moreover, injuries to the nervous system may result in considerable economic impacts to the affected individuals, their families, and the health care system as a whole. To improve quality of life and mitigate direct and secondary impacts of neurological injury, recovering patients may be treated with therapeutic methods designed to improve muscle response and function. One such example of a therapeutic method is functional electrical stimulation therapy (FES-T).

FES-T implements the technique of functional electrical stimulation (FES). FES itself can produce contractions in paretic muscles using a series of short electrical pulses and has been used in the past as a dynamic orthosis that enhances the user's ability to manipulate objects or perform other motor functions only when the stimulation is activated. FES-T builds on this response and focuses on delivering a short-term therapy for promoting neurorecovery. FES-T may include, for example, delivering movement-specific stimulation patterns while the user attempts to perform voluntary functional movements (e.g., object manipulation) over a series of sessions.

To the extent that there exists evidence that therapeutic methods such as FES-T are effective, the response across different muscles and muscle groups to such therapeutic methods can vary and the factors determining muscle responsiveness are poorly understood. For instance, some paralyzed muscles that exhibit no voluntary contractions nonetheless have detectable electrophysiological responses, while others do not.

Even muscles that regain strength during FES-T do so gradually, and changes may not be immediately apparent. Therefore, early determinations of whether a muscle or muscle group will respond to FES-T, to what degree, and how rapidly, are of significant clinical interest.

### SUMMARY

In view of these and other circumstances, the present disclosure provides for methods, systems, and devices to provide point-of-care prediction of muscle responsiveness to FES-T during neurorehabilitation.

In one aspect of the present disclosure, there is provided a portable, hand-held device comprising: a sensor configured to record surface electromyography (sEMG) data for at least one muscle; a memory; and a processor configured to apply predetermined relationships between the sEMG data and reference data stored in the memory, and based on the relationships, generate a predicted recovery profile for the muscle.

In another aspect of the present disclosure, there is provided a method of training a portable device in a functional electrical stimulation therapy (FES-T) program including a first session, a plurality of intermediate sessions, and a last session, the method comprising: prior to the first session, administering an electromyography (EMG) evaluation to a subject and generating a predicted recovery profile for a muscle of the subject; at a beginning of the first session, the plurality of intermediate sessions, and the last session, administering a longitudinal evaluation to the subject; after the last session, generating an actual recovery profile for the muscle; correlating the predicted recovery profile with the actual recovery profile using at least one metric; and based on the correlating, storing reference data in a memory of the portable device.

In another aspect of the present disclosure, there is provided a method of predicting muscle recovery in a functional electrical stimulation therapy (FES-T) program, the method comprising: prior to a first session of the FES-T program, recording surface electromyography (sEMG) data for a muscle using a portable device; identifying correlations between the sEMG data and reference data stored in a memory of the portable device; and generating a predicted recovery profile for the muscle.

Additional aspects of the present disclosure will be set forth in part in the description which follows, and in part will be obvious from the description or may be learned by practice of the present disclosure. The aspects of the present disclosure, and advantages which arise therefrom, may be realized and attained by means of the elements and combinations particularly pointed out in the appended claims and their equivalents.

It is to be understood that both the foregoing general description and the following detailed description are exemplary and explanatory only, and are not restrictive of the present disclosure and claims.

### BRIEF DESCRIPTION OF THE DRAWINGS

These and other aspects of the present disclosure are described with respect to the attached drawings, in which:

FIG. 1 illustrates an exemplary portable device in accordance with various aspects of the present disclosure;

FIG. 2 illustrates an exemplary predictive method in accordance with various aspects of the present disclosure;

FIG. 3 illustrates exemplary feature formulae in accordance with various aspects of the present disclosure;

FIGS. 4A-7 illustrate exemplary sEMG signals in accordance with various aspects of the present disclosure; and

FIG. 8 illustrates an exemplary recovery profile in accordance with various aspects of the present disclosure.

### DETAILED DESCRIPTION

As noted above, early determinations of whether a muscle or muscle group will respond to FES-T, to what degree, and how rapidly, are of significant clinical interest. Without a sufficient understanding of the factors that predict responsiveness to FES-T, and a method to ascertain them in a clinical context at an early stage, it may remain difficult or impossible to appropriately personalize therapy for each patient. Moreover, in the absence of such an understanding and method, therapy time may be spent on muscles that have a low chance of seeing functional improvements. In-patient rehabilitation programs are a strong driver of direct health care costs after neurological injuries, and the resulting pressures on the health care system may lead to progressively shorter hospital stays.

Therefore, there exists a need to tailor in-patient rehabilitation programs to each individual in an evidence-based manner to ensure that the best possible use is made of the limited hospital stay. Similarly, in the chronic stages of injury, there exists financial pressure to maximize outcomes in as few sessions as possible. Predicting the response of each muscle or muscle group to FES-T may enable the development of personalized therapy plans that improve outcomes while making better use of the limited treatment time available to patients and practitioners. This may result in improved therapy efficacy while ensuring efficient use of limited health care resources.

The present disclosure relates to systems, methods, and devices for predicting the responsiveness of a muscle or muscle group to functional electrical stimulation therapy (FES-T).

More particularly, the present disclosure relates to systems, methods, and devices for determining correlation between electrophysiological biomarkers detected during surface electromyography (sEMG) and responsiveness to FES-T and application of the correlation as a predictive tool. The present disclosure provides an overview of the testing and data analysis performed to identify electrophysiological biomarkers that can be detected in sEMG data of a given muscle or muscle group and used to predict the responsiveness of that muscle or muscle group to FES-T. Said another way, the electrophysiological biomarkers can be used to predict the success of FES-T for a particular muscle or muscle group.

The data providing correlations between sEMG and responsiveness to FES-T can be used to create lookup tables or for machine-learning (training) purposes to create a portable, handheld diagnostic device capable of performing sEMG on an individual muscle or muscle group, identify, extract, and/or calculate one or more electrophysical biomarkers from the sEMG data for the individual muscle or muscle group, and predict the FES-T responsiveness of the individual muscle or muscle group based on the electrophysical biomarker(s). Handheld devices in accordance with the present teachings may include EMG electrodes, a microcontroller configured for data acquisition, signal processing, and biomarker computation, and a housing. The housing may include a display to communicate output to the user and/or to solicit input from the user. Exemplary outputs include, but are not limited to: prediction of success (e.g., in yes/no format), likelihood of success (e.g., as a percent chance), expected amount of recovery (e.g., on a strength scale), expected time to first increase (e.g., as a number of sessions), and/or expected time to plateau (e.g., as a number of sessions). The EMG electrodes may be surface or skin electrodes and may be incorporated into a disposable insert to be received by the housing or may be sterilizable and form a permanent part of the housing.

The present disclosure provides systems, methods, and devices which may be used to personalize FES-T for improvement in outcomes and healthcare resource utilization. Certain aspects of the present disclosure provide for analyses to be used in point-of-care screening of patients to predict potential responsiveness of individual muscles or muscle groups to FES-T. Such analyses may be used for many purposes, including but not limited to enabling therapists to quickly and easily screen muscles and predict their response to FES-T, to develop a plan for therapy progression. In some examples, various aspects of the present disclosure may prospectively characterize strength recovery profiles of individual muscles or muscle groups during FES-T, may characterize electrophysiological profiles of impaired muscles at baseline using surface electromyography (sEMG), and/or may identify electrophysiological biomarkers that can predict muscle recovery profiles from baseline sEMG data. These aspects may be implemented by, for example, a handheld device including a sensor configured to record sEMG data for a muscle or muscle group, a memory, and a processor configured to apply predetermined relationships between the sEMG data and reference data stored in the memory, and based on the relationships, generate a predicted recovery profile for the muscle. The relationships may be in the form of algorithms, equations, models, and the like; thus, the processor may be configured to apply the predetermined relationship by applying the algorithms, equations, models, and the like to the data.

As used herein, neurological injuries and neurotrauma may refer to various conditions including but not limited to stroke, spinal cord injury (SCI) and disease, brain injury, peripheral nerve injury, radiculopathy, multiple sclerosis (MS), cerebral palsy, and/or upper motor neuron diseases and injuries. These conditions may affect the function of body parts including but not limited to the upper limbs (e.g., reaching and grasping functions), the lower limbs (e.g., walking and standing balance functions), the trunk (e.g., sitting balance functions), and/or the face.

### Systems and Methods

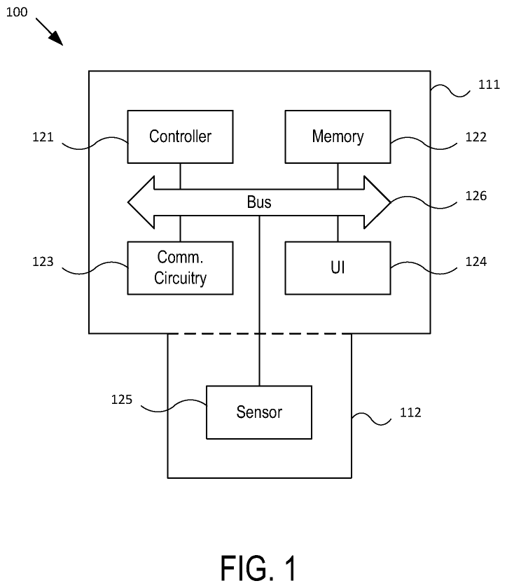

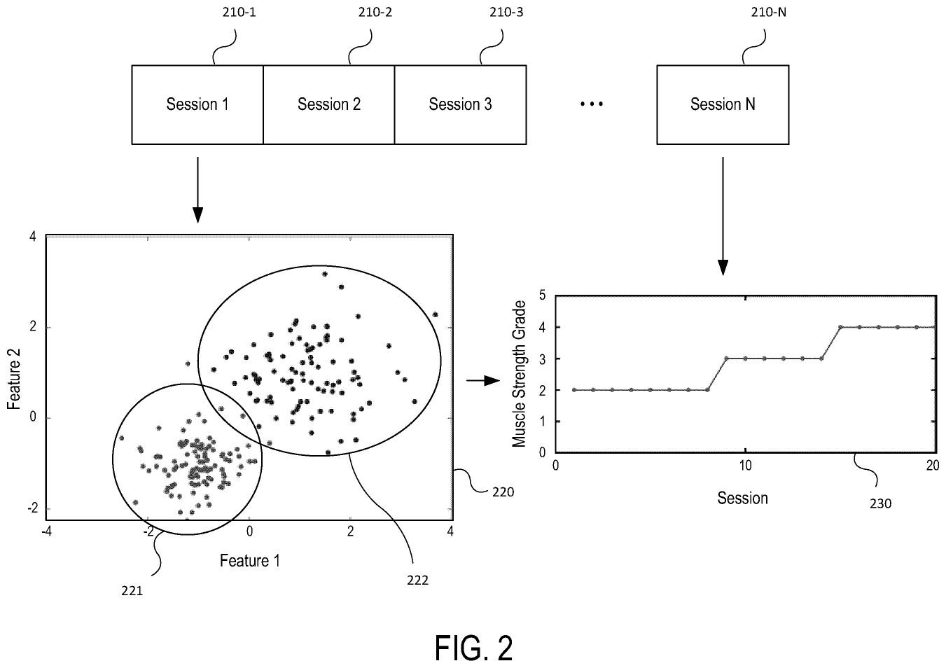

FIG. 1 illustrates an exemplary portable handheld device 100 in accordance with various aspects of the present disclosure. FIG. 2 illustrates an exemplary predictive FES-T method in accordance with various aspects of the present disclosure, which may be facilitated with the portable device 100 of FIG. 1. The portable handheld device 100 may include a base portion 111 and a probe portion 112. Within the portable device 100 are several components, including a controller 121, a memory 122, communication circuitry 123, a user interface 124, and a sensor 125. The internal components may communication with one another via a bus 126. In some examples, the portable device 100 may have a wand-shaped (e.g., having a profile that is generally cylindrical and generally longer in the axial direction than in directions perpendicular to the axial direction) form factor. Alternatively, other form factors are possible and are within the scope of this disclosure. For example, the handheld device may be embodied as a handheld component of a tabletop or cart-based system.

In some implementations, the base portion 111 and the probe portion 112 may be separable, as illustrated in FIG. 1 by a dashed line. For example, the probe portion 112 may be removably attached to the base portion 111 by one or more fasteners and/or communication interfaces (e.g., to facilitate communication between components of the base portion 111 and components of the probe portion 112). The probe portion 112 may thus be removed to facilitate sterilization, to provide modular functionality to the portable device 100, and the like. Additionally or alternatively, the probe portion may be disposable or include a disposable portion to eliminate the need for sterilization between patients. In other implementations, the base portion 111 and the probe portion 112 may form a unitary housing. In either implementation, the portable device 100 may have a wand-shaped form factor to facilitate the targeting of individual muscles. Moreover, in either implementation, the probe portion 112, and possibly a part or all of the base portion 111, may be configured to be covered by a sterile drape. Such configuration may be implemented through the use of one or more fasteners and/or attachment points on the exterior of the probe portion 112 and/or the base portion 111.

The controller 121 includes components configured to control other elements of the portable device 100, to process instructions received from the memory 122 or other sources, to perform various method operations (including but not limited to those described herein), to apply signal processing and/or machine learning algorithms to analyze data (e.g., the sEMG data described below), to perform calculations and/or predictions, and the like. In some examples, the controller 121 may be or include one or more central processing units (CPUs), arithmetic logic units (ALUs), floating-point units (FPUs), or other microcontrollers. The memory 122 includes components configured to store and/or retrieve information. In some examples, the memory 122 may be or include one or more storage elements such as Random Access Memory (RAM), Read-Only Memory (ROM), optical storage drives and/or disks, magnetic storage drives and/or tapes, hard disks, flash memory, removable storage media, and the like.

The communication circuitry 123 includes components configured to allow communication (i.e., transmission and reception) with other components and devices. The communication circuitry 123 may provide physical and/or virtual interfaces and ports for performing wired communication, wireless communications via radio transmission, optical communication via fiber or free electromagnetic radiation, and the like. The communication circuitry 123 may further provide for connections to peripheral devices, such as Universal Serial Bus (USB) devices. The user interface 124 includes components configured to allow interaction with a user, including but not limited to the presentation of information to the user (e.g., via a display, speakers, haptic feedback devices, and the like) and/or the receipt of information from the user (e.g., via a touch screen, microphone, camera or other gesture detection device, physical buttons, soft buttons, and the like). The user interface 124 may include or generate one or more graphical user interfaces (GUIs) and associated elements such as icons, menus, images, and the like. The user interface 124 may be configured to report the results of calculations and/or predictions generated by the controller 121 to the user. Exemplary information output via the user interface 124 includes, but is not limited to: prediction of success (e.g., in yes/no format), likelihood of success (e.g., as a percent chance), expected amount of recovery (e.g., on a strength scale), expected time to first increase (e.g., as a number of sessions), and/or expected time to plateau (e.g., as a number of sessions). Exemplary information input via the user interface 124 includes, but is not limited to, identification of the muscle being analyzed, type of injury or disease, and/or severity of injury or disease.

The sensor 125 includes components configured to sense and/or detect various signals. In implementations where the sensor 125 collects readings directly from the subject, the sensor 125 may be a single contact sensor or an array of contact sensors such as a recording electrode (e.g., a surface or skin electrode) and may or may not be used with a conductive gel. The type, size, and material of electrode used may vary, for example based on muscle location and size. Examples of surface electrodes include metal plate or limb electrodes, suction cup/Welsh cup electrodes, multipoint electrodes, and adhesive electrodes and floating electrodes. In embodiments where electrode size/shape/type might vary due to physical body parameters, the device may be configured to incorporate a disposable sensor element, such that an appropriate type of sensor element can be selected dependent upon the location of the muscle measurements being taken. The interchangeability/replaceability/disposability of the sensor also would potentially eliminate the need for sterilization of the device between patients. The sensor 125 may be configured to measure and record the muscle response (e.g., from individual muscles or muscle groups) of the subject (e.g., sEMG data). In some implementations, the sensor 125 may further be configured to apply a stimulus (e.g., electrical pulses for FES) to the subject. The sensor 125 may further include components such as filters (e.g., bandpass filters, low-pass filters, high-pass filters, etc.), amplifiers, converters (e.g., analog-to-digital converters (ADCs), digital-to-analog converters (DACs), etc.), samplers, and the like.

Individual components of the portable device 100 may be implemented via dedicated hardware components, by software components, by firmware, or by combinations thereof. Hardware components may include dedicated circuits such as application-specific integrated circuits (ASICs), field-programmable gate arrays (FPGAs), and the like. Software components may include software modules stored in memory (e.g., the memory 122), instructions stored on a non-transitory computer readable medium (e.g., the memory 122 or an external memory) and executed by a processor (e.g., the controller 121), remote instructions received from an external source (e.g., via the communication circuitry 123), and the like.

In use, the probe portion 112 of the device 100 is placed in contact with an area of the subject's body containing the target muscle, such that the sensor 125 is brought near the target muscle and may obtain readings therefrom. The readings may be obtained by applying electrical pulses to the body at a location near the target muscle (e.g., by an electrode of the device 100) and measuring changes in voltage in the target muscle, which may be caused by contractions and extensions responsive to the electrical pulses. The pulses may be applied, and the readings taken, while the subject is voluntarily contracting the target muscle to a predetermined degree, including a full contraction. The voltage readings may be filtered, amplified, and sampled as described above, and may be stored as an amplitude signal as a function of time and/or in the form of extracted features (e.g., in the memory 122).

### Data for Algorithmic Training

As shown in FIG. 2, to create a data set to establish correlation between electrophysiological biomarkers in sEMG data and responsiveness to FES-T, a plurality of subjects were followed through FES-T. Each subject (e.g., a patient) with a neurological injury or disease underwent a longitudinal muscle strength evaluation during the course of an FES-T program including a series of N FES-T sessions, including a first session 210-1 and a last session 210-N with a plurality of intermediate sessions 210-2, 210-3, etc. therebetween (collectively referred to as “sessions 210”). N is an integer and, in some examples may be 20-40 sessions 210, although fewer or more sessions 210 may be conducted. The longitudinal evaluation may take place at the beginning of every session 210, and in some examples may be administered by the treating therapist. In some examples, the longitudinal evaluation may be based on manual muscle testing (MMT), which assigns each muscle a grade between 0 (e.g., no visible voluntary contraction) to 5 (e.g., normal).

In an exemplary MMT evaluation, the subject performs standardized movements to isolate the activity of the target muscle. For upper limb evaluations, the target muscle may be the deltoid muscles, elbow flexors, elbow extensors, wrist extensors, extensor digitorum communis, opponens pollicis, flexor pollicis longus, flexor pollicis brevis, finger flexors, finger abductors, and/or dorsal interossei. For lower limb evaluations, the target muscle may be the bilateral quadriceps, hamstrings, dorsiflexors, and/or plantar flexors. For trunk evaluations, the target muscle may be the rectus abdominis and/or erector spinae. For facial evaluations, the target muscle may be the zygomaticus major and/or the orbicularis oculi. These lists are not exhaustive, and other target muscles are within the scope of the present disclosure.

The set of muscles tested is not limited to those muscles described above and may include less than all muscles described above. In some examples, the set of muscles may be individualized for the subject, for example based on the subject's therapy plan and/or goals. Any muscle receiving stimulation or expected to later receive stimulation may be tracked for the duration of the longitudinal evaluation. Additionally, muscles may be added to the tracking group as the therapy plan evolves.

The course of FES-T may be individualized according to the subject's impairment and/or goals. In each session 210, multichannel FES-T may be applied in combination with task-specific motor training to promote improvement of muscle function for activities of daily living. A session 210 may last approximately one hour and may be repeated four to five days per week. Over the course of the sessions 210, a subject may progress through movement sequences directed to regaining natural, unassisted voluntary movement in the affected area (e.g., an extremity). The session 210 may be conducted according to a protocol which specifies the sequence of muscle stimulation required to precipitate a natural movement and practice a functional task (e.g., picking up an object, reaching forward or sideways, grasping and retrieving an object, and so on). The conductor of the session 210 (e.g., the treating therapist) may control the amplitude of stimulation for each channel, decide when to engage the stimulation for each muscle, and/or balance the contributions of manual support, voluntary movement, and FES.

Actual recovery profiles of individual muscles may be generated at the conclusion of the longitudinal evaluation (i.e., after session 210-N) and, in some implementations, at various intermediate points during the longitudinal evaluation. The recovery profile of a muscle may be described by the absolute improvement in function (e.g., the number of MMT grades gained), the speed of improvement in function (e.g., the time of first increase in MMT grade), and/or the course of improvement in function (e.g., the time at which the final MMT grade is achieved). In some examples, “recovery” may be deemed an increase in MMT of one grade or more over the course of treatment. FIG. 2 illustrates one exemplary recovery profile 230, which shows measurements of the muscle strength grade (measured using MMT) vs. time (measured in terms of number of sessions). The speed and/or course of improvement may be measured in terms of the number of FES-T sessions 210 for that muscle, and not necessarily in terms of the number of days since the first session 210-1. These descriptors may be examined through descriptive statistics, applied to the entire dataset and to sub-groupings by muscle and by baseline characteristic (e.g., baseline MMT grade). Regression models may be used to interpret each descriptor based on muscle (a categorical variable) and baseline characteristic (an ordinal variable).

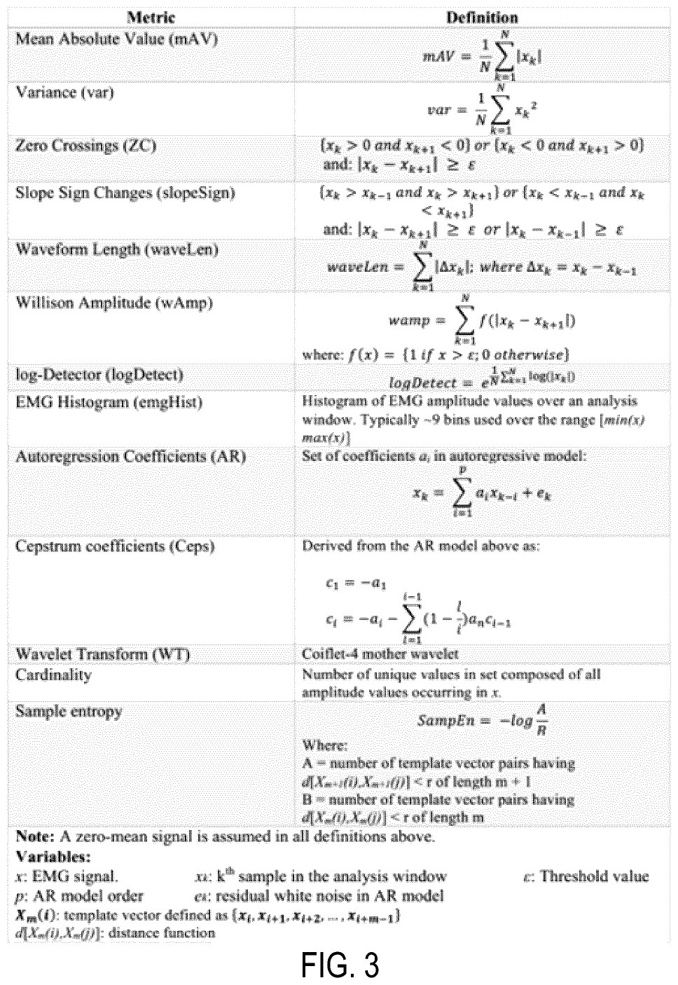

Prior to the first session 210-1, the subject undergoes an EMG evaluation to characterize electrophysiological profiles of individual muscles; for example, to produce a set of feature values. The set of feature values may be combined with corresponding feature values from other subjects, thereby to produce a feature correlation plot 220. The feature correlation plot 220 correlates two different sEMG features, which may be mean absolute values, zero crossings, slope sign changes, waveform length, Willison amplitude, variance, v-order, log-detection, EMG histogram, peak amplitude, autoregression coefficients, median frequency, Cepstrum coefficients, wavelet transform coefficients, maximum fractal length, cardinality, sample entropy, and/or the estimated number of active motor units. Exemplary formulae for generating the feature correlation plot 220 are illustrated in FIG. 3. The EMG evaluation may, using baseline sEMG recordings, identify distinct electrophysiological properties in impaired muscles during voluntary contraction. This may rely on the application of clustering algorithms in a feature space derived from the sEMG signals (e.g., as shown in the feature correlation plot 220).

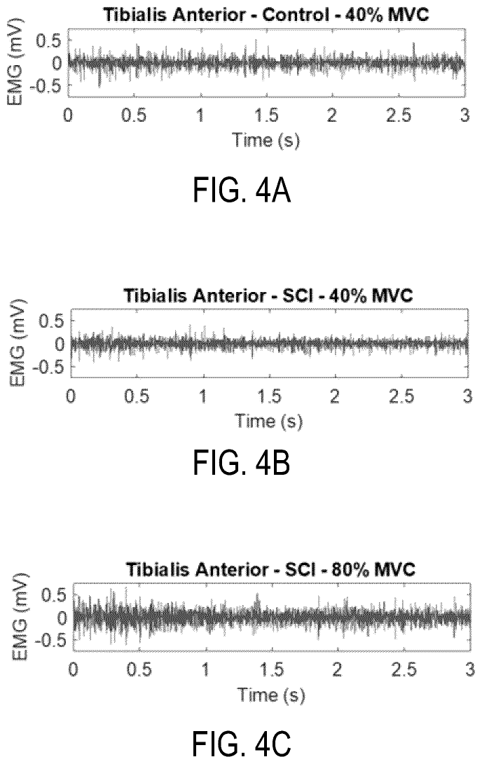

The sEMG features are used to provide additional insight into signal properties and provide better characterization than signal amplitude alone, as illustrated in FIGS. 4A-C. FIGS. 4A-C respectively illustrate EMG response signals vs. time for the tibialis anterior of a control subject at 40% of maximal voluntary contraction (MVC), a subject with SCI at 40% of MVC, and the subject with SCI at 80% MVC. Superficially, it may be difficult to distinguish the response signals visually based solely on their respective amplitudes and/or root-mean-square (RMS) values. However, differences between the signals become apparent when viewed in terms of sEMG features. The present disclosure is not limited to 40% and 80% of MVC, and in practical implementations may use data for any predetermined percentage of MVC.

FIG. 4A results in a normalized RMS (nRMS) value of 0.048, a zero crossing (ZC) value of 820, a slope sign change (SS) value of 290, and a Willison amplitude (wAmp) of 1492. FIG. 4B results in an nRMS value of 0.041, a ZC value of 839, an SS value of 185, and a wAMP value of 2363. FIG. 4C results in an nRMS value of 0.0671, a ZC value of 997, an SS value of 599, and a wAmp value of 5049. In this example, a large difference in wAmp between the control subject and the SCI subject at the same contraction amount (i.e., between FIG. 4A and FIG. 4B) is observable and evidence of impaired tibialis anterior strength. Likewise, several metrics evidence the intra-subject strength of contraction within the SCI subject (i.e., between FIG. 4B and FIG. 4C), which may be used to provide information about muscle unit responsiveness to FES-T.

Returning to FIG. 2, the feature correlation plot shows two clusters 221 and 222. While FIG. 2 shows a 2-feature correlation, in practice additional features may be simultaneously correlated or analyzed. In one example, six features may be correlated. Features may additionally be combined to create a smaller or larger set of new features. The clustering algorithms may categorize muscles based on the sEMG data into three groups; for example: unimpaired muscles, muscles with impaired upper motor neuron function but intact lower motor neuron function, and muscles with impaired lower motor neuron function. The number of groups is not limited to three, and in some implementations may be any number greater than or equal to two.

The sEMG evaluation may be performed by or with the portable device 100, which may be the same device or a different device as the one used in obtaining the readings as described above. The conductor may use the portable device 100 to collect sEMG data from all muscles that will be tracked in the longitudinal strength assessment. For each muscle, the subject may be asked to perform a number (e.g., three) of maximal voluntary contractions and a number (e.g., three) of half-maximal contractions. The portable device 100 may apply a bandpass filter between 10 hertz (Hz) and 1 kilohertz (kHz) to the sEMG data, amplify it (e.g., via an amplifier), and sample it at 5 kHz (e.g., via sampling circuitry). The operations of filtering, amplification, and sampling may be performed by various components of the portable device 100, including the controller 121, the sensor 125, and/or other circuitry. For the half-maximal contractions, the data acquisition may be guided by a visual threshold overlaid over a real-time visualization of the EMG signal. The half-maximal contractions may be held for 5 seconds, and the portable device 100 may retain the middle 3 seconds for analysis.

The collected data may be processed (e.g., by the controller 121) to extract one or more of the sEMG features described above. Additionally or alternatively, the data may be pre-processed at another location and then loaded (e.g., from the memory 122) for further processing. Collectively, these features reflect different aspects of the signal, including amplitude, frequency, and complexity characteristics. The processing may include cluster identification to determine clusters with sufficient separation to serve as the basis for the predictive modeling (which will be described in more detail below). In one example, the clustering analysis may proceed in three stages. in some examples, the predictive modeling may proceed from the sEMG features without a clustering stage. The following stages are exemplary, and the present disclosure encompasses clustering analyses with additional or alternate stages.

Stage one may include feature selection and dimensionality reduction. As uninformative features may reduce clustering performance, the stage is configured to select or reformulate features to improve the feature set's ability to describe relevant clusters in the data. Stage one may include methods such as principal component analysis (PCA), filter methods of feature selection (i.e., methods to remove features that do not display sufficient variability across the data set, as measured for example by their variance of a χ² quality-of-fit test against a normal distribution), and/or wrapper methods of features selection (e.g., sequential forward selection, in which features are added progressively based on which feature most improves the result).

Stage two may include clustering using one or more clustering algorithms. Distances between data points may be computed using the standardized Euclidean distance. The number of clusters may be varied over a relevant range (e.g., 0-10) to optimize the fit to the data. Stage two may result in a relatively small number of clusters, corresponding to the different possible patterns of injury (e.g., unimpaired, denervated muscle, partial upper motor neuron damage with intact lower motor neuron, and so on).

Stage three may include cluster quality evaluation. An initial evaluation of the clustering results may use a predetermined coefficient (e.g., a Silhouette coefficient) to measure how close each data point is to other points within its cluster versus points in other clusters. After the initial evaluation has been completed for the characterization of electrophysiological phenotypes and for the identification of electrophysiological biomarkers (described in more detail below), a subset of clustering methods may be reevaluated to optimize performance directly on the recovery prediction metrics. If, in the initial evaluation, more than one clustering method produces acceptable results, the one with the highest value for the predetermined coefficient may be retained.

Using data from the longitudinal evaluation and the EMG evaluation, the conductor may use the systems, methods, and devices described herein to identify electrophysiological biomarkers that can predict muscle recovery profiles from baseline sEMG data.

In one example, if successful clusters were identified in the EMG evaluation, regression models are used to predict recovery profile metrics (e.g., the absolute improvement in function, speed of improvement in function, and/or course of improvement in function as described above). These predictions may be based on combinations of the cluster, muscle, and MMT grades. Multinomial logistic regression and multilinear regression models may be used for the regression. Additionally or alternatively, non-linear regression models may be used by applying machine learning algorithms (e.g., support-vector machine (SVM), random forests, naive Bayes, neural network and so on). In another example, machine learning approaches may be used to predict recovery profiles directly based on one or more of the sEMG features described above. This example may be performed even where the EMG evaluation did not identify successful clusters.

The recovery profile predictions may be evaluated (e.g., correlated with one another) using one or more metrics. Exemplary metrics include: errors (i.e., differences between predicted and actual data) in the number of MMT grades gained, the time of the first increase in MMT grade, and the time at which the final MMT grade was achieved; Pearson correlation between predicted and actual profiles over time, for example where predicted profiles have been constructed using an interpolation based on the predicted number and timing of MMT grade changes; and accuracy in categorizing muscles as responders with a nonzero MMT grade gain and non-responders with a zero MMT grade gain.

Regression and machine learning algorithms may be evaluated using a leave-out-subject-out cross-validation (LOSOCV) process, in which a model is trained on data from all data sets (e.g., participants) except one, and then tested on the muscles of the remaining data set. Each data set is iteratively omitted in turn, and the results are averaged to obtain the final performance metric.

The identification operations (i.e., the generation of predictions using regression models and/or machine learning algorithms) may be performed by the portable device 100, for example using the controller 121. This identification operation may produce a prediction of a muscle strength recovery profile using only baseline sEMG data and MMT score. A prediction may be evaluated from the LOSOCV process on its accuracy in distinguishing responder from non-responder muscles and on the Pearson correlation between the correlation between the predicted and actual recovery profiles (e.g., by determining whether the accuracy and/or Pearson correlation exceeds predetermined thresholds). In one example, the prediction may be defined as successful if the LOSOCV evaluation results in both greater than 90% accuracy in distinguishing responder from non-responder muscles, and greater than 0.5 average Pearson correlation between the predicted and actual recovery profiles.

Thus, the method may identify electrophysiological biomarkers that are capable of predicting muscle recovery profiles from baseline sEMG data, by linking the sEMG features or clusters derived from feature correlation plot 220 to the corresponding recovery profile 230. The results may then be displayed to the operator by the portable device 100, for example using components of the user interface 124.

### EXAMPLES

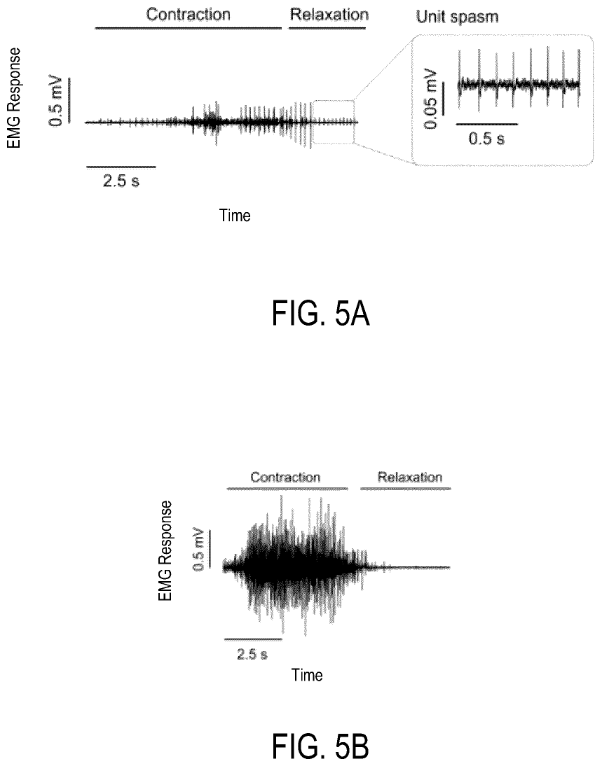

The above systems and methods were utilized to collect data from several participants. FIGS. 5A-8 illustrate exemplary data from one such participant. FIGS. 5A and 5B illustrate exemplary EMG response signals for the participant, where FIG. 5A shows the EMG response of a particular muscle (i.e., the flexor pollicis brevis) of the participant prior to FES-T (e.g., before the first session 210-1) and FIG. 5B shows the EMG response of the muscle after FES-T (e.g., after the final session 210-N). For this particular participant, the number of sessions N is 27.

FIGS. 5A and 5B both illustrate the EMG signal profile when the participant was asked to voluntary contract the muscle for a period of time (as illustrated, approximately 5-10 s) and then relax the muscle. FIG. 5A shows a weak muscle response, as evidenced by the small value (as illustrated, approximately 0.3 mV) of the EMG signal during the contraction phase. FIG. 5A also shows unit spasms (inset) during the relaxation phase, which are regularly repeated involuntary muscle spasms. FIG. 5B shows a stronger muscle response, as evidenced by the large value (as illustrated, approximately 2 mV) of the EMG signal during the contraction phase. FIG. 5B does not show the presence of any unit spasms during the relaxation phase, which is another indication of muscle recovery.

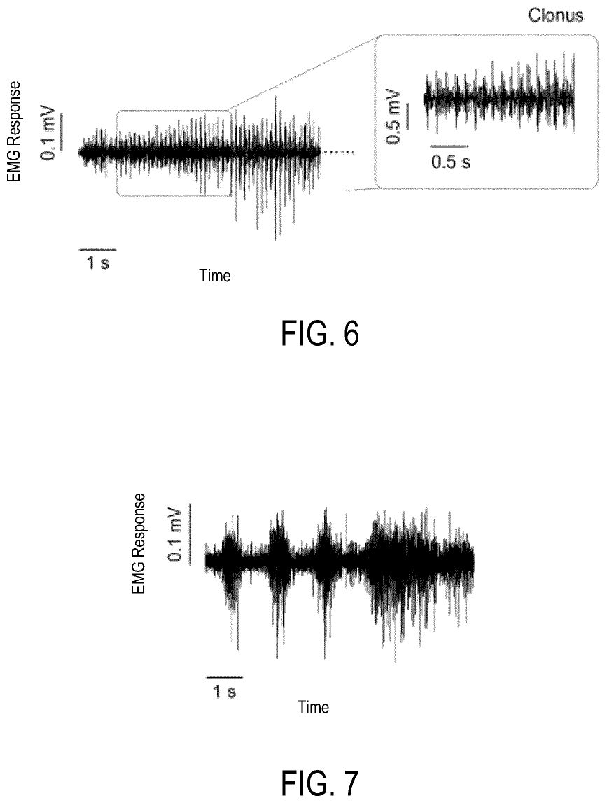

FIG. 6 illustrates the presence of clonus during contractions. The particular illustration of FIG. 6 corresponds to the flexor digitorum superficialis of the participant. Clonus is uncontrollable, repetitive involuntary muscle movement which occur rapidly. Clonus may be another indication of muscle weakness. As shown in FIG. 6 (inset), the participant experienced clonus resulting in involuntary contractions approximately five to eight times per second. FIG. 7 illustrates a clonic spasm of the participant in the triceps brachii.

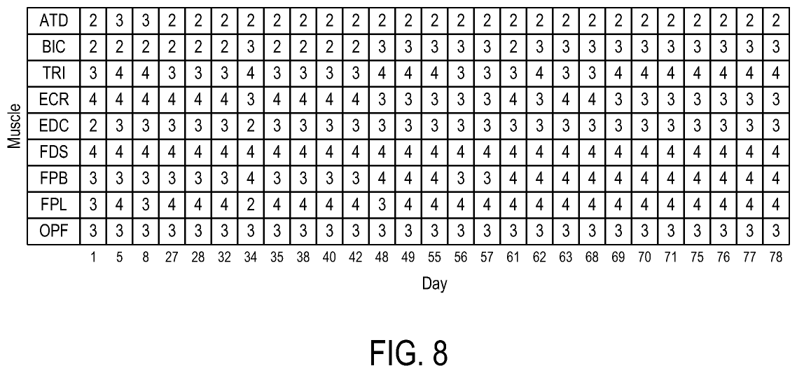

FIG. 8 illustrates an exemplary overall recovery profile from the participant, generated from a series of measurements similar to those used to obtain the graphs of FIGS. 5A-7. FIG. 8 illustrates the MMT grades of the participant for nine muscles over the course of 27 sessions spanning 78 days. The particular muscles evaluated were the anterior deltoid (ATD), biceps brachii (BIC), triceps brachii (TRI), extensor carpi radialis (ECR), extensor digitorum communis (EDC), flexor digitorum superficialis (FDS), flexor pollicis brevis (FPB), flexor pollicis longus (FPL), and opponens pollicis (OPF). Each evaluated muscle was assigned an MMT grade at the beginning of each session. Each individual muscle may be characterized and/or presented to the conductor in a manner similar to the recovery profile 230 illustrated in FIG. 2. From FIG. 8, it can be seen that certain muscles increased in MMT grade over the course of FES-T, whereas other muscles did not increase over the course of FES-T. In particular, six muscles exhibited responsiveness to FES-T: the biceps brachii, the triceps brachii, the extensor digitorum communis, the flexor pollicis brevis, and the flexor pollicis longus.

In the example illustrated in FIGS. 5A-8, it was shown that, by applying a k-nearest neighbours classifier to two sEMG features (2nd coefficient of a 4th-order autoregressive model and 2nd cepstrum coefficient) on 72 muscles from 9 individuals, muscles could be classified as FES-T responders with an F1-score of 0.70 (precision=0.67, recall=0.73) using a LOSOCV evaluation, or an F1-score of 0.73 (precision=0.7, recall=0.76) using a leave-one-muscle-out evaluation.

### CONCLUSION

The exemplary systems and methods described herein may be performed under the control of a processing system executing computer-readable codes embodied on a computer-readable recording medium or communication signals transmitted through a transitory medium. The computer-readable recording medium may be any data storage device that can store data readable by a processing system, and may include both volatile and nonvolatile media, removable and non-removable media, and media readable by a database, a computer, and various other network devices. The system can involve connected hardware components, such as processors and non-transitory memory.

Examples of the computer-readable recording medium include, but are not limited to, read-only memory (ROM), random-access memory (RAM), erasable electrically programmable ROM (EEPROM), flash memory or other memory technology, holographic media or other optical disc storage, magnetic storage including magnetic tape and magnetic disk, and solid state storage devices. The computer-readable recording medium may also be distributed over network-coupled computer systems so that the computer-readable code is stored and executed in a distributed fashion. The communication signals transmitted through a transitory medium may include, for example, modulated signals transmitted through wired or wireless transmission paths. For example, embodiments described herein can provide a system for predicting muscle recovery in a functional electrical stimulation therapy (FES-T) program can involve transmitting communication signals corresponding to a predicted recovery profile for the muscle using a transceiver and/or communication network. The system can have at least one processor that identifies relationships or correlations between surface electromyography (sEMG) data for at least one muscle and reference data stored in memory, and, based on the relationships or correlations, generates the predicted recovery profile for the muscle.

The above description and associated figures teach the best mode of the invention and are intended to be illustrative and not restrictive. Many embodiments and applications other than the examples provided would be apparent to those skilled in the art upon reading the above description. The scope should be determined, not with reference to the above description, but instead with reference to the appended claims, along with the full scope of equivalents to which such claims are entitled. It is anticipated and intended that future developments will occur in the technologies discussed herein, and that the disclosed systems and methods will be incorporated into future embodiments. In sum, it should be understood that the application is capable of modification and variation.

All terms used in the claims are intended to be given their broadest reasonable constructions and their ordinary meanings as understood by those knowledgeable in the technologies described herein unless an explicit indication to the contrary is made herein. In particular, the use of the singular articles such as “a,” “the,” “said,” etc. should be read to recite one or more of the indicated elements unless a claim recites an explicit limitation to the contrary.

The Abstract is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1. A portable, hand-held device, comprising:
a sensor configured to record surface electromyography (sEMG) data for at least one muscle;
a memory; and
a processor configured to:
apply predetermined relationships between the sEMG data and reference data stored in the memory, and
based on the relationships, generate a predicted recovery profile for the muscle.

2. The device according to claim 1, wherein the processor is configured to identify correlations by applying a machine learning algorithm to the sEMG data.

3. The device according to claim 2, wherein the machine learning algorithm is trained by:
applying a clustering algorithm to the sEMG data, thereby to assign the at least one muscle to a category, and
based on the category or directly from the sEMG data, determining at least one electrophysiological biomarker, and
associating the electrophysiological biomarker with a likelihood of muscle recovery.

4. The device according to claim 2, wherein the sensor is configured to record the sEMG data for the at least one muscle over for at least one session of functional electrical stimulation therapy (FES-T).

5. The device according to claim 4, wherein the plurality of sessions is 20-40 sessions.

6. The device according to claim 2, wherein the machine learning algorithm is configured to categorize the at least one muscle into one of a predetermined number of groups.

7. The device according to claim 2, wherein the processor is configured to extract a plurality of sEMG features from the sEMG data.

8. The device according to claim 7, wherein respective ones of the plurality of sEMG features are selected from the group consisting of mean absolute value, zero crossings, slope sign changes, waveform length, Willison amplitude, variance, v-order, log-detection, EMG histogram, peak amplitude, autoregression coefficients, median frequency, Cepstrum coefficients, wavelet transform coefficients, maximum fractal length, cardinality, sample entropy, and an estimated number of active motor units.

9. The device according to claim 7, wherein the machine learning algorithm is configured to analyze the sEMG data in a feature space using at least two of the plurality of sEMG features.

10. The device according to claim 2, wherein the sEMG data includes first data corresponding to a maximal voluntary contraction (MVC) and second data corresponding to a predetermined percentage of MVC.

11. The device according to claim 2, further including a filter configured to apply a bandpass filter to the sEMG data, an amplifier configured to amplify the filtered sEMG data, and sampling circuitry configured to sample the filtered and amplified sEMG data.

12. The device according to claim 2, wherein the machine learning algorithm is configured to generate the predicted recovery profile using a regression model.

13. The device according to claim 1, wherein the reference data includes information relating to a relationship between at least one electrophysiological biomarker and a likelihood of muscle recovery.

14. The device according to claim 1, further comprising a housing configured to contain the sensor, the memory, and the processor.

15. The device according to claim 14, wherein the housing comprises a base portion containing the memory and the processor, and a probe portion containing the sensor, wherein the probe portion is configured to removably attach to the base portion, wherein the probe portion is configured to be covered by a sterile drape.

16. (canceled)

17. (canceled)

18. The device according to claim 1, further comprising a user interface configured to present information to a user and/or receive information from the user, wherein the user interface includes at least one of a display, a touch screen, a speaker, a microphone, a camera, a haptic feedback device, a Physical device, or a soft button.

19. (canceled)

20. The device according to claim 1, further comprising communication circuitry configured to provide wired or wireless communication with an external device.

21. The device according to claim 1, wherein the at least one muscle is selecting from the group consisting of upper limb muscles, lower limb muscles, trunk muscles, and face muscles.

22. A method of training a portable device in a functional electrical stimulation therapy (FES-T) program including a first session, a plurality of intermediate sessions, and a last session, the method comprising:
prior to the first session, administering an electromyography (EMG) evaluation to a subject and generating a predicted recovery profile for a muscle of the subject;
at a beginning of the first session, the plurality of intermediate sessions, and the last session, administering a longitudinal evaluation to the subject;
after the last session, generating an actual recovery profile for the muscle;
correlating the predicted recovery profile with the actual recovery profile using at least one metric; and

23-45. (canceled)

46. A system for predicting muscle recovery in a functional electrical stimulation therapy (FES-T) program, the system comprising:
a memory; and
at least one processor coupled to the memory, wherein the processor:
identifies relationships or correlations between surface electromyography (sEMG) data for at least one muscle and reference data stored in the memory; and
based on the relationships or correlations, generates a predicted recovery profile for the muscle; and
transmits the predicted recovery profile over a network, or stores the predicted recovery profile in the memory.

47-67. (canceled)
